(19) United States
(12) Patent Application Publication
(10) Pub. No.: US 2025/0177607 A1
(43) Pub. Date: Jun. 5, 2025

(54) 3D PRINTED CONDUCTIVE BIOPOLYMER FOR CARDIAC TISSUE ENGINEERING

(71) Applicant: Board of Regents, The University of Texas System, Austin, TX (US)

(72) Inventors: Sylvia Lucia Natividad-Diaz, El Paso, TX (US); Britanny L. Stark, El Paso, TX (US)

(21) Appl. No.: 19/035,447

(22) Filed: Jan. 23, 2025

Related U.S. Application Data
(63) Continuation-in-part of application No. 18/733,235, filed on Jun. 4, 2024.
(60) Provisional application No. 63/655,448, filed on Jun. 3, 2024.
(60) Provisional application No. 63/512,192, filed on Jul. 6, 2023.

(51) Int. Cl. A61L 27/20

(52) U.S. Cl. CPC A61L 27/20; A61L 2430/20

(57) ABSTRACT

An engineered material includes a conductive biopolymer including a plurality of layers, each of which includes methacrylated hyaluronic acid conjugated with at least one of 3-thiophene acetic acid and with poly(3-thiophene) acetic acid; and myocytes. The myocytes can include iPSC-derived cardiomyocytes. The myocytes can be substantially aligned in single lines. The single lines can be approximately parallel to one another and approximately normal to planes defined by the plurality of layers. A method of 3D printing the layers is also described.

### CROSS-REFERENCE TO RELATED APPLICATIONS

Referring to the application data sheet filed herewith, this application claims a benefit of priority from co-pending U.S. Ser. No. 18/733,235, filed Jun. 4, 2024 (attorney docket number UTEP2023-008) which in turn claims a benefit of priority under 35 U.S.C. 119 (c) from both U.S. Ser. No. 63/512,192, filed Jul. 6, 2023 (attorney docket number UTEP2023-008-PROV) and U.S. Ser. No. 63/655,448 filed Jun. 3, 2024 (attorney docket number UTEP2023-008-PROV-2), the entire contents of all of which are hereby expressly incorporated herein by reference for all purposes.

### STATEMENT AS TO RIGHTS TO INVENTIONS MADE UNDER FEDERALLY-SPONSORED RESEARCH OR DEVELOPMENT

This invention was made with government support under 1SC2GM144164-01 awarded by NIH/NIGMS. The government has certain rights in the invention.

### BACKGROUND

Hyaluronic Acid is the most abundant glycosaminoglycan in the body and the main component for many tissues. It contributes to tissue hydrodynamics, movement and proliferation of the cells. Additionally, hyaluronic acid is chemically versatile, which allows it to be modified easily while remaining biocompatible.

Referring to FIG. 1, methacrylated hyaluronic acid (Me-HA) adds a UV cross linkable component to hyaluronic acid, making it possible to 3D print hydrogels for tissue engineering applications. A problem with this technology has been how to make and use such a 3D printable, UV curable hydrogel conductive for electronic or even electrical purposes.

There is a need for electron-hole conductive 3D printable hydrogels for tissue engineering. Previous approaches to producing conductive methacylated hyaluronic acid have not been entirely successful and research continues for alternative methacylated hyaluronic acid based materials that are conductive. Therefore, what is required are better approaches to making methacrylated hyaluronic acid (Me-HA) into a conductive material.

Meanwhile, in other fields, thiophene is known as a colorless heterocyclic compound. In medicine, thiophene is known as an anti-inflammatory, an anti-psychotic, and an anti-arrhythmic. In material science, thiophene is known for its use in compositions that are inhibitors of corrosion in metals and for use in components for light-emitting diodes.

Referring to FIG. 2, when treated with an oxidizing agent, thiophene electrons are delocalized via a process called doping. Doping intentionally introduces impurities into a compound to change its electrical properties. The delocalized electrons move along the polymer backbone forming a charged unit called a bipolaron.

Heretofore, the requirement of making methacrylated hyaluronic acid (Me-HA) into a conductive material referred to above has not been fully met. In view of the foregoing, there is a need in the art for a solution that solves this problem.

### SUMMARY

There is a need for the following embodiments of the present disclosure. Of course, the present disclosure is not limited to these embodiments.

Embodiments of the present disclosure can include a composition of matter comprising a conductive biopolymer comprising a plurality of layers, each of the plurality of layers comprising methacrylated hyaluronic acid conjugated with at least one of 3-thiophene acetic acid and with poly(3-thiophene) acetic acid; and myocytes. The myocytes can include iPSC-derived cardiomyocytes. The myocytes can be substantially aligned in single lines. The single lines can be approximately parallel to one another and approximately normal to planes defined by the plurality of layers. Other embodiments can include a method of 3D printing the a conductive biopolymer comprising the plurality of layers.

In this project, a novel conjugate between Me-HA and 3-thiopheneacetic acid (3TAA) has been developed to make an electrically conductive hyaluronic acid polymer and solid hydrogel. The 3TAA contains thiophene, a chemical compound that can be doped to introduce electrical conductivity. Overall, conjugating 3TAA to Me-HA will create an electrically conductive hydrogel for cardiac and neural tissue engineering applications. This electrically conductive hyaluronic acid hydrogel also has applications in the electronic devices industry. Since the electrically conductive hyaluronic acid polymer can be crosslinked with UV and LAP, this material can also be printed into a solid customized shape with the fabrication technique called 3D stereolithography (SLA) printing.

According to another embodiment of the present disclosure, a composition of matter comprises: methacrylated hyaluronic acid conjugated with 3-thiophene acetic acid or with poly(3-thiophene) acetic acid.

According to another embodiment of the present disclosure, a method comprises Fischer esterification of methacrylated hyaluronic acid with 3-thiophene acetic acid in the presence of an acid activator to produce methacrylated hyaluronic acid conjugated with 3-thiopheneacetic acid.

According to another embodiment of the present disclosure, a method comprises Fischer esterification of methacrylated hyaluronic acid with poly(3-thiophene) acetic acid in the presence of an acid activator to a produce methacrylated hyaluronic acid conjugated with poly(3-thiophene) acetic acid.

These, and other, embodiments of the present disclosure will be better appreciated and understood when considered in conjunction with the following description and the accompanying drawings. It should be understood, however, that the following description, while indicating various embodiments of the present disclosure and numerous specific details thereof, is given for the purpose of illustration and does not imply limitation. Many substitutions, modifications, additions and/or rearrangements may be made within the scope of embodiments of the present disclosure, and embodiments of the present disclosure include all such substitutions, modifications, additions and/or rearrangements.

### BRIEF DESCRIPTION OF THE DRAWINGS

The drawings accompanying and forming part of this specification are included to depict certain embodiments of the present disclosure. A clearer concept of the embodiments described in this application will be readily apparent by referring to the exemplary, and therefore nonlimiting, embodiments illustrated in the drawings (wherein identical reference numerals (if they occur in more than one view) designate the same elements).

FIG. 1 is a chemical structural representation of methacrylated hyaluronic acid, appropriately labeled “PRIOR ART.”

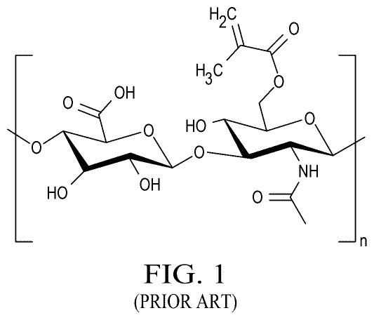

FIG. 2 is a chemical structural representation of thiophene, appropriately labeled “PRIOR ART.”

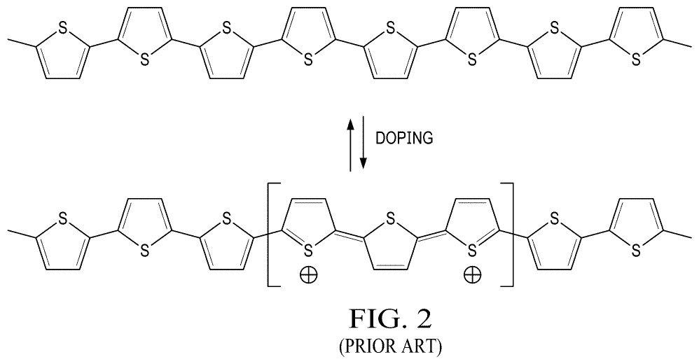

FIG. 3 is a chemical reaction representation of an example of a reaction of methacrylated hyaluronic acid and thiophene using carbonyldiimidazole (CDI).

FIG. 4 is a chemical reaction representation of an example of a reaction of methacrylated hyaluronic acid and thiophene using sulfuric acid.

FIG. 5 is a presentation of preliminary results including doping of 3TAA with iodine to introduce electrical conductivity properties.

FIGS. 6A-6B are NMR Spectra for (a) MeHA and (b) MeHA 3TAA.

FIGS. 7A-7B are statistical comparison in conductivity measurements for (a) 3TAA and (b) MeHA 3TAA. n=4 (**** p<0.0001).

FIGS. 8A-8B are amplitude sweep for (a) MeHA and (b) MeHA 3TAA hydrogels.

FIG. 9 is statistical comparison in storage and loss modulus between MeHA and MeHA 3TAA. n=48 (**** p<0.0001).

FIG. 10 is MTT Assay statistical comparison in absorbance between MeHA and MeHA 3TAA.

FIGS. 11A-11L are immunostaining of AC-16 in MeHA and MeHA 3TAA.

FIG. 12 is cell count of live cells in MeHA and MeHA 3TAA.

FIGS. 13A-13H are immunostaining of iPSC cardiomyocytes in MeHA and MeHA 3TAA.

FIG. 14 is statistical comparison of storage and loss modulii between 3D-printed MeHA 3TAA and non-3D printed MeHA 3TAA.

FIG. 15 is statistical comparison of storage and loss modulii between MeHA and 3D Printed MeHA 3TAA.

FIGS. 16A-16B are bright field images of 3D printed MeHA 3TAA with iPSC cardiomyocytes substantially aligned in single lines.

FIGS. 17A-17B are images of immunostaining for day 5 of the 3D printed iPSC cardiomyocytes encapsulated in MeHA 3TAA hydrogels.

### DETAILED DESCRIPTION

Embodiments presented in the present disclosure and the various features and advantageous details thereof are explained more fully with reference to the nonlimiting embodiments that are illustrated in the accompanying drawings and detailed in the following description. Descriptions of well-known materials, techniques, components and equipment are omitted so as not to unnecessarily obscure the embodiments of the present disclosure in detail. It should be understood, however, that the detailed description and the specific examples are given by way of illustration only and not by way of limitation. Various substitutions, modifications, additions and/or rearrangements within the scope of the underlying inventive concept will become apparent to those skilled in the art from this disclosure.

Embodiments of this disclosure include an electrically conductive hyaluronic acid polymer that can be cross-linked with UV light and lithium phenyl-2,4,6-trimethylbenzoylphosphinate (LAP) to form a solid hydrogel. Embodiments of this disclosure include methacrylate hyaluronic acid conjugation with 3-thiopheneacetic acid.

Embodiments of this disclosure can achieve the conjugation with various chemistries. For example, the reaction chemistry can be driven by CDI (carbonyldiimidazole). As another example, the reaction chemistry can be driven by H₂SO₄ (sulfuric acid). As another example, the reaction chemistry can be driven by 1-Ethyl-3-(3-dimethylaminopropyl) carbodiimide (EDC). As another example, the reaction chemistry can be driven by hydroxybenzotriazole (HOBT)

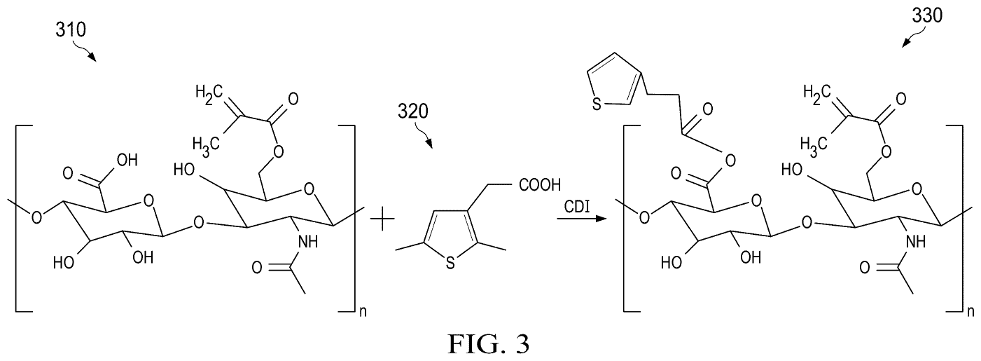

Referring to FIG. 3, methacrylated hyaluronic acid 310 is reacted with 3-thiopheneacetic acid 320 in the presence of CDI (carbonyldiimidazole) as an acid activator to produce methacrylated hyaluronic acid conjugated with 3-thiophene acetic acid 330.

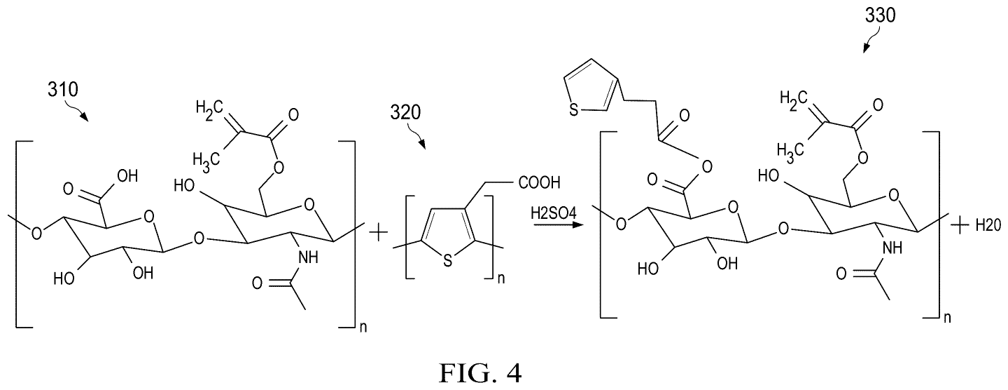

Referring to FIG. 4, methacrylated hyaluronic acid 310 is reacted with 3-thiopheneacetic acid 320 in the presence of H₂SO₄ (sulfuric acid) as an acid activator to produce methacrylated hyaluronic acid conjugated with 3-thiophene acetic acid 330.

A non-mutually exclusive alternative to conjugating with 3-thiopheneacetic acid (3TAA) is an initial polymerization of a source of 3TAA, and subsequent doping with Ferric Chloride or sulfuric acid, to make poly(3-thiophene) acetic acid. Embodiments of this disclosure can achieve this P3TAA conjugation with various chemistries. For example, this reaction chemistry can be driven by CDI (carbonyldiimidazole). As another example, this reaction chemistry can be driven by H₂SO₄ (sulfuric acid). As another example, this reaction chemistry can be driven by 1-Ethyl-3-(3-dimethylaminopropyl) carbodiimide (EDC). As another example, this reaction chemistry can be driven by hydroxybenzotriazole (HOBT).

### EXAMPLES

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### LIST OF REAGENTS:

Methacrylated Hyaluronic Acid
   Sigma-Aldrich, 100 mg-100-150 kDa, SKU #CC327-2
3-thiopheneacetic Acid
   Sigma-Aldrich, 10 g, 142.18 Da, SKU #220639-10G
Pure iodine
   Sigma-Aldrich, 5 g, SKU #207772-5G
Carbonyldiimidazole (CDI)
   Sigma-Aldrich, 5 g, SKU #115533-5G
   Generic Protocol:
3-thiopheneacetic acid doped with iodine
   Dissolve 3-thiopheneacetic acid in 10 mLs DI water with 2 mg of iodine
   Stir for 24 hours at 350 rpms.

### Example 1

In this example, methacrylate hyaluronic acid is conjugated with 3-thiopheneacetic acid in the presence of CDI (carbonyldiimidazole). Once 3TAA is doped, add solution into 100 mg of Methacrylate Hyaluronic Acid, and 1 mg of carbonyldiimidazole (CDI) as a carboxylic acid activator. Then, stir for 24 hours at 350 rpms. Then filter using a membrane of 8 kDa for 24 hours against DI water.

In an alternative to this example, methacrylate hyaluronic acid is conjugated with 3-thiopheneacetic acid in the presence of H₂SO₄ (sulfuric acid). In an alternative to this example, methacrylate hyaluronic acid is conjugated with 3-thiopheneacetic acid in the presence of 1-Ethyl-3-(3-dimethylaminopropyl) carbodiimide (EDC). In an alternative to this example, methacrylate hyaluronic acid is conjugated with 3-thiopheneacetic acid in the presence of hydroxybenzotriazole (HOBT).

### Example 2

A non-mutually exclusive alternative to conjugating with 3-thiopheneacetic acid (3TAA) is an initial polymerization of a source of 3TAA, and subsequent doping with Ferric Chloride or sulfuric acid, to make poly(3-thiophene) acetic acid. Then add the P3TAA solution into 100 mg of Me-HA, and 1 mg of CDI as a catalyst. Then, stir for 24 hours at 350 rpms. Finally, filter using a membrane of 8 kDa for 24 hours against DI water.

In an alternative to this example, methacrylate hyaluronic acid is conjugated with P3TAA in the presence of H₂SO₄ (sulfuric acid). In an alternative to this example, methacrylate hyaluronic acid is conjugated with P3TAA in the presence of 1-Ethyl-3-(3-dimethylaminopropyl) carbodiimide (EDC). In an alternative to this example, methacrylate hyaluronic acid is conjugated with P3TAA in the presence of hydroxybenzotriazole (HOBT).

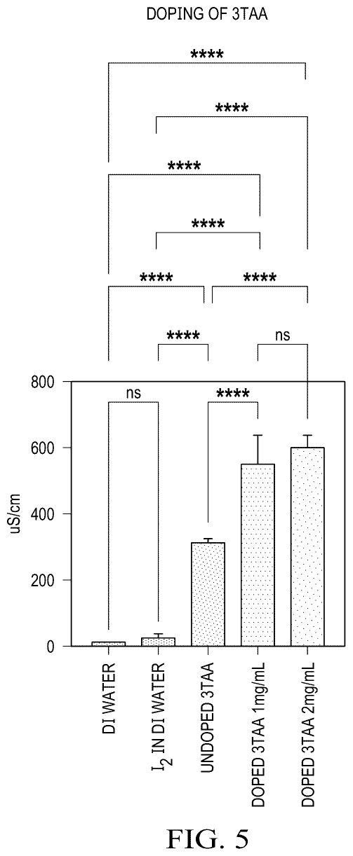

Referring to FIG. 5, a statistically significant difference in conductivity uS/cm was found between doped 3TAA and undoped 3TAA, DI water, and iodine in DI water. This indicates that 3TAA can be electrically conductive, which when conjugated into Me-HA will in turn, make the hydrogel conductive. Conductivity measurements taken with a Dynamic Light Scattering system (DLS)

A practical application of an embodiment of the present disclosure that has value within the technological arts is conjugating 3TAA to Me-HA to create an electrically conductive hydrogel for cardiac and neural tissue engineering applications. Further, embodiments of the present disclosure are useful in electronic devices (such as are used for the purpose of wearables for example hearing aids or eyeglasses), or in conjunction with an implantable (such as are used for the purpose of prosthesis or the like. There are virtually innumerable uses for embodiments of the present disclosure, all of which need not be detailed here.

Embodiments of the present disclosure can be cost effective and advantageous for at least the following reasons. Embodiments that include conjugating 3TAA to Me-HA create an electrically conductive hydrogel for cardiac and neural tissue engineering applications. This electrically conductive hyaluronic acid hydrogel also has applications in the electronic devices industry, especially implantable devices. Since the electrically conductive hyaluronic acid polymer can be crosslinked with UV and LAP, embodiments can be printed into a solid customized shape, such as with the fabrication technique called 3D stereolithography (SLA) printing. Embodiments provide stable, biocompatible conductive hydrogel products. Embodiments of the present disclosure improve quality and/or reduce costs compared to previous approaches.

Key terms and abbreviations include: Hyaluronic Acid (HA), Methacrylated hyaluronic acid (Me=HA), 3-thiophene acetic acid (3TAA), poly(3-thiophene) acetic acid (3TAA) Carbonyldiimidazole (CDI), Sulfuric Acid (H₂SO₄), 1-Ethyl-3-(3-dimethylaminopropyl) carbodiimide (EDC), hydroxybenzotriazole (HOBT), Lithium phenyl-2,4,6-trimethylbenzoylphosphinate (LAP), stereolithography (SLA), and Ultraviolet light (UV).

### Example 3

This example includes development of a conductive hyaluronic acid-based polymer for cardiac tissue engineering.

### Introduction

Acute Myocardial Infarction (AMI) severely disrupts the intricate electrical signaling system of the heart due to the nonconductive fibrotic scar tissue that appears after an AMI. This disruption extends its adverse influence to the remaining viable cardiomyocytes, causing electrical uncoupling, and consequently, ventricular dysfunction. The repercussions of this disruption are far-reaching, potentially leading to arrhythmias and, ultimately, heart failure.

Addressing this formidable challenge, the development of a conductive biopolymer emerges as a promising solution. Research has shown that conductive biomaterials have the potential to enhance cardiac function after implantation, offering a glimmer of hope in the quest to mitigate the effects of AMI. However, these potential breakthroughs have been impeded by the complexity of manufacturing processes, which have yet to achieve clinical feasibility.

In embodiments of this disclosure, a Methacrylated Hyaluronic Acid (MeHA) hydrogel (Sigma) will be conjugated with 3-thiopheneacetic acid to create a semi-synthetic conductive polymer for cardiac tissue engineering. Hyaluronic Acid, an abundant glycosaminoglycan of the extracellular matrix, is known for its chemical versatility and inherent robustness. Moreover, when adding a Methacrylate group to HA, the hydrogel can be photopolymerized using a photoinitiator.

In order to add conductivity for the intricate domain of cardiac tissue engineering, embodiments introduce 3-thiophencacetic acid into the MeHA matrix. Thiophene, a heterocyclic compound celebrated for its low cytotoxicity can undergo chemical conjugations with case. It possesses the unique capability of being rendered conductive through a process known as doping, adding a layer of versatility to the polymer. This polymer will also contain the photo initiator LAP (Sigma-Aldrich) to be later optimized for 3D SLA printing.

Embodiments can include the combination of esterification technique and 3D printing methodology, with the result of developing a conductive biopolymer for cardiac tissue engineering. Embodiments provide the tissue engineering field with an easily replicable, conductive polymer that faithfully mimics the conductive attributes of native myocardium, offering a promising pathway for cardiac health restoration.

### Materials and Methods

### Methacrylated Hyaluronic Acid and LAP

The hydrogel used in this study is Methacrylated Hyaluronic Acid (MeHA) (Sigma), characterized by a molecular weight falling within the range of 100-150 kDa, with a degree of substitution in the range of 20-25%.

To facilitate the crosslinking process, the photoinitiator Lithium Phenyl-2,4,6-trimethylbenzoylphosphinate (LAP) (Sigma) was used. This photoinitiator attaches to the free radical in the MeHA and allows the hydrogel to crosslink at a UV wavelength of 405 nm.

### Doping of 3-Thiopheneacetic Acid

For conductivity grafting into the MeHA matrix, the 3-thiopheneacetic acid (3TAA) (Sigma) molecule was used. This compound has a molecular weight of 142.18 Da. To introduce the impurities that initiate the doping of 3TAA, pure Iodine (Sigma) was added.

A total of 12 mg of 3TAA was dissolved in deionized (DI) water, with 2 mg of pure Iodine. This solution was allowed to mix over a 24-hour period.

### MeHA and 3TAA Conjugation

After the doping process, the 10 mL 3TAA/I₂ solution is mixed with 100 mg of lyophilized MeHA. To catalyze the interaction, 1 mg 1,1′-Carbonyldiimidazole (CDI) (Sigma) is introduced. This is allowed to react for 24 hours while continuously mixing using a platform shaker (IBI Scientific).

Subsequently, the solution undergoes filtration using an 8 kDa dialysis membrane (ThermoFisher) for another 24 hours against DI water, to remove any unreacted molecules. This results in a homogenous clear mixture that can be UV-cross linkable using a photoinitiator.

### Nuclear Magnetic Resonance (NMR) Spectroscopy

For chemical analysis of the MeHA 3TAA hydrogel, Proton NMR was conducted across three different batches of the hydrogel. The instrument used was the Avance III HD NanoBay NMR Spectrometer (Bruker-Spectrospin). This NMR is equipped with a 400 MHz magnet, and a 5 mm PA BBO probe.

For sample preparation, all batches were lyophilized at −80C for a period of 3 days, and later resuspended in 99.8%-grade Deuterium Oxide (D2O) (Sigma). The solution was placed in 400 MHz 5 mm diam. Wilmad NMR tubes (Sigma).

All the data obtained was analyzed using MNova NMR Software (Mestrelab Research, Spain).

Dynamic Light Scattering (DLS) system

For all conductivity measurements, the Zetasizer Advance Dynamic Light Scattering (DLS) (Malvern Panalytical, United Kingdom) system was used.

The 3TAA/I₂ solution was tested three times, across three different batches, to ensure robustness and accuracy. This solution was juxtaposed with the 3TAA in solution with DI water, as well as DI water alone.

Additionally, the MeHA 3TAA hydrogel was tested three times, also across three different batches, and compared with MeHA and DI water alone.

### Mechanical Testing Using Rheology

To determine the mechanical properties of the hydrogel after its conjugation with 3TAA, rheology was conducted using a SmartPave 92 Dynamic Shear Rheometer (Anton Paar, Austria).

An amplitude sweep of the MeHA and MeHA 3TAA hydrogels was performed six times each to establish the Linear Viscoelastic Range (LVER) of the material. The shear strain rate of the test was set to a range spanning from 0.1 to 10%.

After establishing the LVER of the materials, a frequency sweep was conducted to determine the complex modulus of the materials. Using the amplitude sweep data, the shear strain set for the frequency sweep was 1%, and the frequency was set to 1.99 Hz. This test involved three different crosslinked hydrogels across three different batches.

### MTT Assay

To establish biocompatibility of the MeHA 3TAA hydrogel, an MTT Assay (R&D Systems) was conducted. This assay was done with the AC-16 [Millipore, SCC109] human cardiomyocyte cell line, cultured in Dulbecco's modified Eagle's medium F-12 containing 2 mmol/L L-glutamine, 12.5% FBS, and 1× penicillin-streptomycin. This experiment was done across a period of 3, 7 and 14 days in culture with MeHA and MeHA 3TAAA, using round bottom 96-well plates and a seeding density of 1×10⁵ cells/mL.

The cells were stained after each period with MTT, and allowed to react for 24 hours, after which they were washed using the provided detergent from the manufacturer for another 24 hours.

After staining, a plate reader was used to measure the absorbance of the solution at 590 nm.

### Immunostaining

To further establish biocompatibility of the hydrogel, an immunostaining process was performed after culturing AC-16s [Millipore, SCC109] for a period of 3, 7 and 14 days. Additionally, human-induced Pluripotent Stem Cell (hiPSC) derived cardiomyocytes were cultured for a period of 7 and 14 days in RPMI B27. AC-16s were cultured at a seeding density of 1×10⁵ cells/cm², and iPSCs cardiomyocytes at a seeding density of 3×10⁶ cells/mL.

Both cell lines were cultured in a Transwell (ThermoFisher) and a 12-well plate in both MeHA and MeHA 3TAA. The cells were fixed with PFA, and the hydrogels were permeabilized with 0.1% Triton (ThermoFisher). Two different antibodies were used for staining, the primary antibody was CTnT, and the secondary antibody was F-Actin 555 and Alexa Fluor 488. During the first antibody stain, 2% BSA was added to prevent non-specific binding of CTnT. Before imaging, DAPI is added to the hydrogels to stain the nucleus of the cells. For a comprehensive overview, Table 1 shows the antibodies and fluorescent markers, along with the color they fluoresce, for all stains used in this procedure.

TABLE 1
Antibodies used for immunofluorescence staining.

| Antibody | Concentration | | Color | Purpose |
|---|---|---|---|---|
| CTnT | 1:200 | μL | Green | Cardiac Troponin T binds tropomyosin to regulate |
| | | | | calcium binding and contractility of striated muscle |
| | | | | present in cardiac cells. |
| F-Actin | 1:200 | μL | Red | Binds to the N-terminal region of the actin filaments |
| | | | | present in muscle cells |
| DAPI | 2 | drops/mL | Blue | Binds to adenine-thymine-rich regions in DNA. |
| | | | | Effectively showing cell nuclei. |

After staining, the hydrogels were imaged using a Leica Thunder 3D Imager (Leica Microsystems, Germany).

Furthermore, an image analysis of the cell circumference was taken on the AC-16 images, to quantify the difference in cell size between cells encapsulated in both hydrogels. This analysis was done using a Python algorithm developed by the SND lab.

hiPSC Cardiomyocytes Beat Rate

Videos of the spontaneously beating hiPSC deviced cardiomyocytes were taken using a Leica Thunder 3D Imager with a DLS 9000GT camera. The beat rate of the hiPSC derived cardiomyocytes encapsulated in the MeHA 3TAA hydrogel was calculated using the average displacement over time. This calculation was made using MTrackJ from ImageJ (NIH). From the average displaced, the beat per minute was calculated based on the peaks of displacement.

### Statistical Analysis

All quantitative measurements were performed in triplicate samples, unless otherwise stated. All values are expressed as mean±Standard Deviation (SD). A Oneway ANOVA with a post-hoc Tukey test, and a 2way ANOVA were used to compare treatment groups; and p<0.05 was used to assess statistical significance using GraphPad Prism.

### Results and Discussion

### NMR Spectroscopy Shows 3TAA Conjugation to MeHA

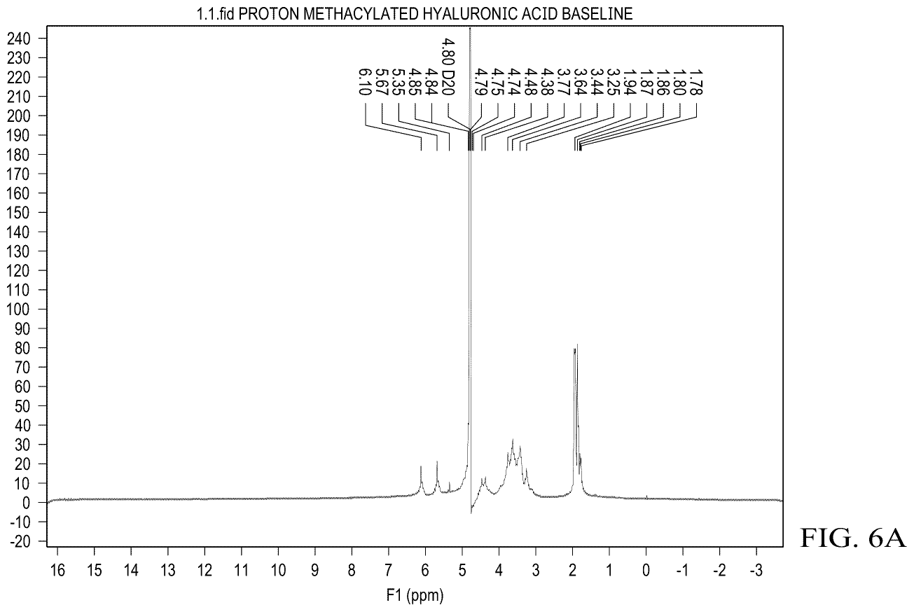

The Proton NMR data shows the conjugation of a thiophene group to the MeHA compound. FIG. 6A shows the NMR of the MeHA as it appears in literature, and FIG. 6B shows the MeHA 3TAA NMR spectra. In FIG. 6A, the peaks for the backbone of Hyaluronic Acid can be seen between 4 and 3 ppm. The peaks that appear between 6.5 ppm and 5.5 ppm, and between 2 ppm and 2.5 ppm (labelled 1, and 2 in the figure) show the methacrylate group in MeHA.

In FIG. 6B, the same peaks from the MeHA can be seen (labelled in the figure as HyA, 1 and 2), but there are an additional four peaks present. These peaks show between 7 ppm and 7.5 ppm, and at 9 ppm, and they belong to the protons present in 3-thiopheneacetic acid.

The presence of the 3TAA peaks indicate that the compound has attached to the MeHA effectively.

### DLS Data Shows Improved Conductivity

The conductivity measurements taken with the DLS system show a significant increase in conductivity after doping the 3TAA with Iodine. For a comprehensive overview, Table 2 delineates the average conductivity measurements, presenting a clear contrast between 3TAA, doped 3TAA, and DI water as control.

TABLE 2
Average conductivity measurements for doped 3TAA

| | | Standard | Coefficient of |
|---|---|---|---|
| Compound | Conductivity (μS) | Deviation (SD) | Variation (%) |
| DI water | 4.58 | 2.31 | 50.5 |
| I₂ in DI water | 27.4 | 5.60 | 20.4 |
| Undoped 3TAA | 317.8 | 3.69 | 1.2 |
| 1 mg/mL 3TAA | 603.8 | 37.0 | 6.13 |
| 2 mg/mL 3TAA | 553 | 86.02 | 15.6 |

Moreover, the conductivity measurements for the MeHA 3TAA hydrogel showcased a similar trend, revealing a significant surge in conductivity when compared to the MeHA material in isolation. This insight can be seen in Table 3, which shows the average conductivity measurements for MeHA and MeHA 3TAA.

TABLE 3
Average conductivity measurements for MeHA and MeHA 3TAA

| | | Standard | Coefficient of |
|---|---|---|---|
| Compound | Conductivity (μS) | Deviation (SD) | Variation (%) |
| MeHA | 1333.7 | 61.6 | 4.62 |
| MeHA 3TAA | 2499 | 119.8 | 4.80 |

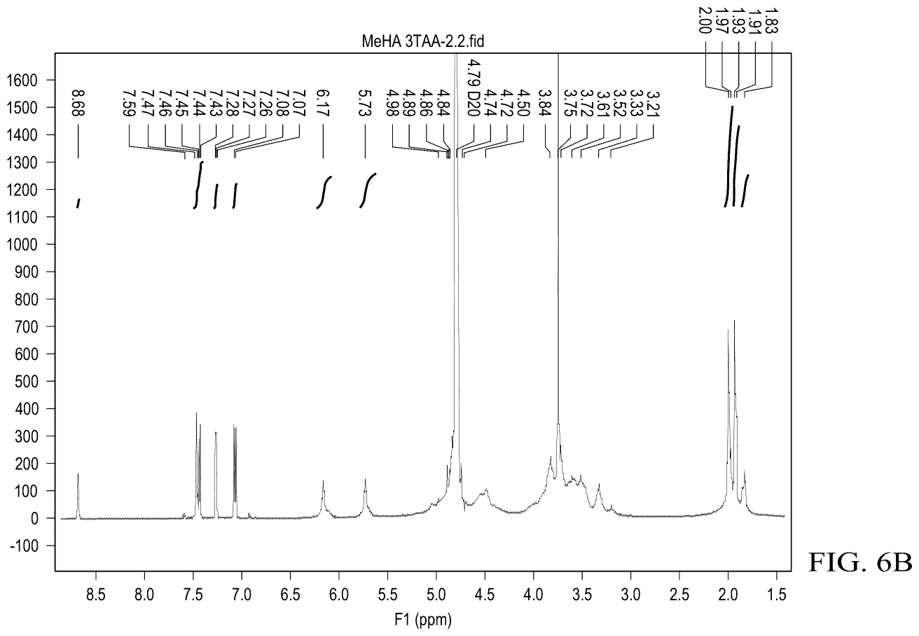

To offer a perspective on these findings, FIG. 7A shows a statistical comparison in conductivity measurements between DI water, I₂ in DI water, and two different concentrations of 3TAA in DI water. In a similar fashion, FIG. 7B shows a statistical comparison between DI water, MeHA and MeHA 3TAA. These analyses paint a compelling picture of the improvement in conductivity achieved through the chemical synthesis of MeHA and 3TAA.

Mechanical Properties after 3TAA Conjugation

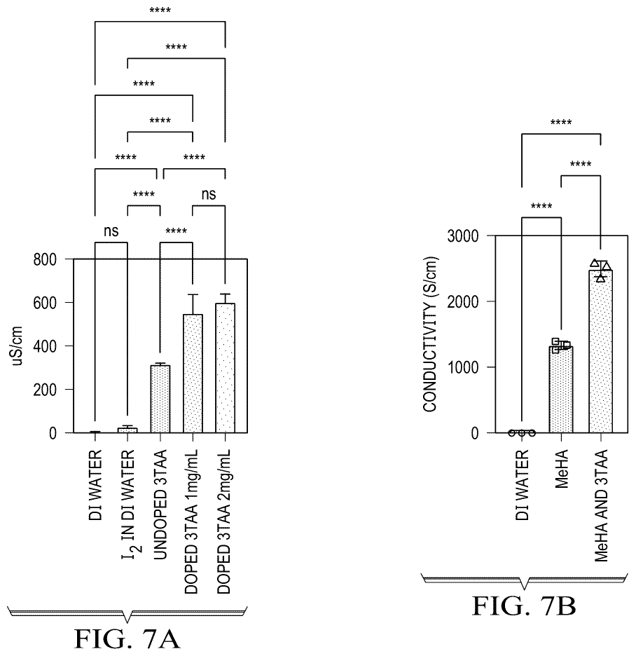

The amplitude sweep of both hydrogels revealed a slight difference in the LVER of the material. After conjugation, the MeHA 3TAA shows a slight decline in complex modulus compared to the unconjugated MeHA. FIGS. 8A-8B show the amplitude sweep for both MeHA (FIG. 8A) and MeHA 3TAA (FIG. 8B).

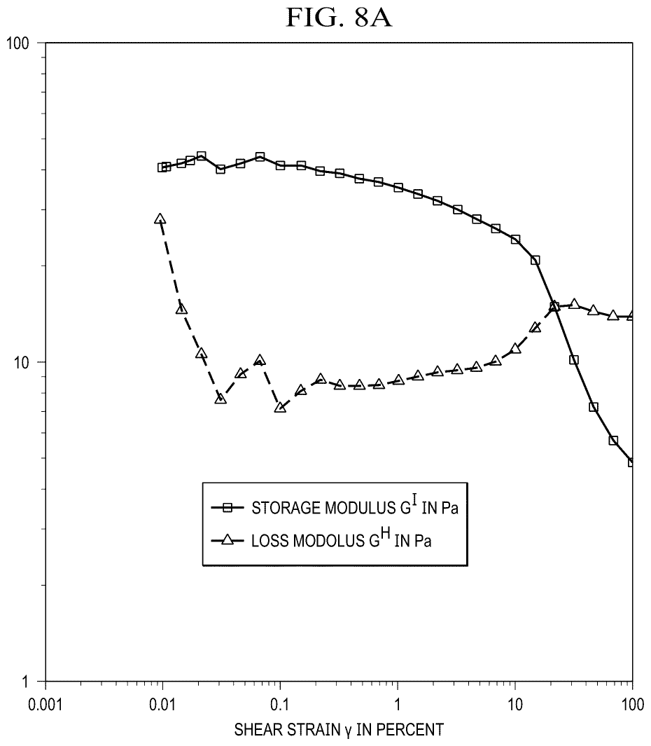

In the case of the frequency sweep, the data shows the conjugated MeHA 3TAA has a significantly lower complex modulus compared to that of the MeHA. In Table 4 the average storage and loss modulus for both hydrogels are shown. FIG. 9 shows a statistical comparison between storage and loss modulus data, with a statistically significant difference.

TABLE 4
Average storage and loss modulus for MeHA and MeHA 3TAA

| | | Measurement | Standard | Coefficient of |
|---|---|---|---|---|
| Hydrogel | Modulus | (Pa) | Deviation (SD) | Variation (%) |
| MeHA | Storage Modulus | 34.9 | 2.56 | 10.2 |
| MeHA 3TAA | Storage Modulus | 26.7 | 6.9 | 25.9 |
| MeHA | Loss Modulus | 9.03 | 0.63 | 6.70 |
| MeHA 3TAA | Loss Modulus | 14.7 | 3.40 | 23.1 |

Biocompatibility Tests Show No Changes after 3TAA Conjugation

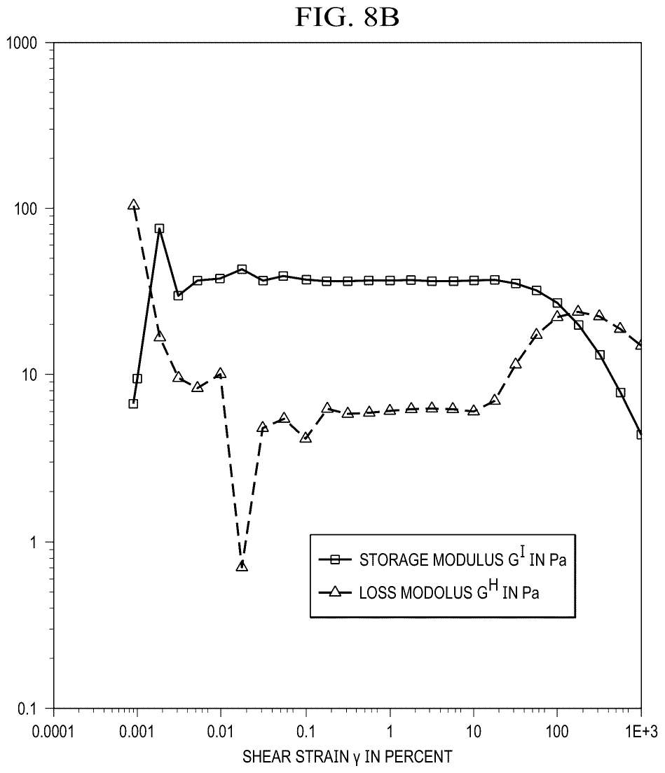

MTT Assays show that there is no statistically significant difference between the AC-16 cells that are in 3D culture in the MeHA and in the MeHA 3TAA. FIG. 10 shows the results of the absorbance data obtained after days 3, 7 and 14 in-culture. Both hydrogel cultures show similar absorbance across time, which indicates that the MeHA 3TAA does not affect cell viability.

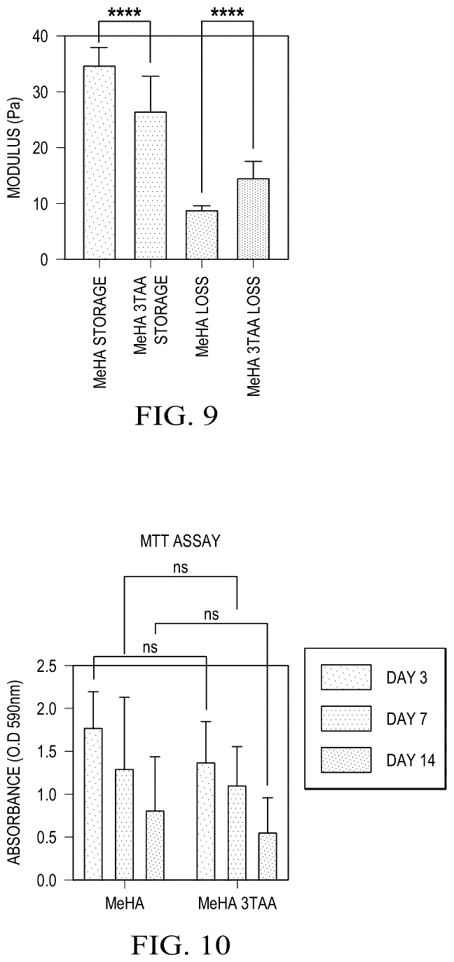

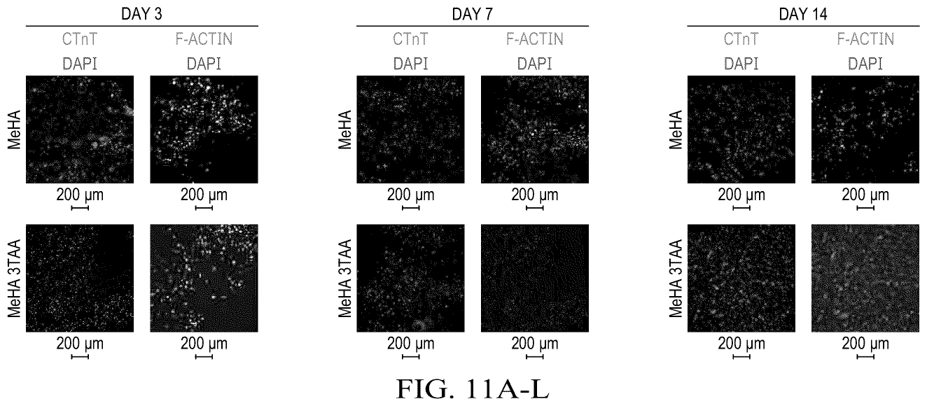

When it comes to immunostaining, the AC-16s show no difference between the MeHA and the MeHA 3TAA cultures, as can be seen in FIGS. 11A-11L. Furthermore, FIG. 12 shows the quantitative analysis, which demonstrates that the cell circumference is not significantly different between both hydrogels. This means that the 3TAA group conjugation to MeHA does not affect the original biocompatibility properties of the hydrogel.

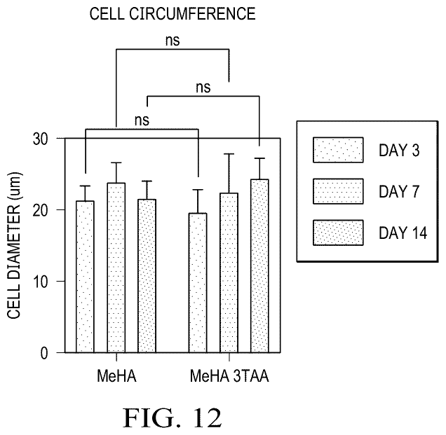

Similarly, the iPSC cardiomyocytes followed the same trend. No difference can be seen between the MeHA and MeHA 3TAA hydrogels, as portrayed by FIGS. 13A-13H.

Beat rate analysis shows iPSC cardiomyocytes are able to beat in the conjugated hydrogel

The average displacement calculated for the movement of the cardiomyocytes was 945.27 μm (±5.94 SD). The displacement indicates the beat rate of the cardiomyocytes. The beat calculation using average displacement has been used in literature before. The average beat per minute was calculated to be 60 bpm (±4.88 SD).

### Conclusions

Embodiments address the challenge posed by AMI, a condition that disrupts the electrical signaling system of the heart, which leads to arrythmias, ventricular dysfunction and ultimately, heart failure. The nonconductive fibrotic scar tissue that arises after an AMI creates a barrier against the normal electrical propagation in the heart, leaving viable cardiomyocytes in a state of electrical uncoupling and dysfunction. Because of this, the need for a conductive biopolymer emerged as a therapeutic tool to resolve the electric impedance of the fibrotic scar tissue in the native myocardium.

Conductive biomaterials hold great promise in enhancing cardiac function after implantation. However, much remains to be improved in terms of the conductive properties of the material, and clinical feasibility. Embodiments synthesize a MeHA hydrogel with a doped solution of 3TAA, resulting in a semi-synthetic conductive polymer for cardiac tissue engineering.

The 3TAA can be doped in its monomer form, without the need to be polymerized. Furthermore, when conjugated with MeHA, the conductivity of the hydrogel was significantly improved, while retaining the original biocompatible properties of the hydrogel. Embodiments can conjugate 3TAA and MeHA to create a photo-cross linkable conductive biopolymer for cardiac tissue engineering. Furthermore, conjugating 3TAA with hydrogel MeHA does not alter the hydrogel MeHA biocompatibility properties.

### Example 4

This example includes optimizing photopolymerization of conductive biopolymer for 3D SLA printing of myocardial tissue materials.

### Introduction

The advent of 3D printing technologies has ushered in a new era of rapid prototyping, streamlined design processes, and unparalleled manufacturing precision. These remarkable attributes make 3D printing an ideal candidate for applications in tissue engineering, where the demand for patient-specific designs and prototyping holds immense promise.

However, despite the tremendous progress in 3D printing, the choice of materials for this revolutionary technique remains a formidable challenge. For applications in tissue engineering, these biomaterials must possess a unique blend of qualities, encompassing bioresorbable, bioactivity, biocompatibility, and mechanical robustness. Considering these challenges, embodiments of this disclosure capitalize on Me-HA/thiophene composites, harnessing their potential to advance the field of cardiac tissue engineering.

3D printing capabilities, coupled with the conductivity of the Me-HA/Th hydrogel, enable the creation of tissue constructs characterized by precise shapes and structures. Cardiac tissue can be meticulously tailored to match the anatomical features of specific regions within the heart. Embodiments can provide, at least in part, a platform for patient-specific treatments and region-specific modeling/manufacturing.

### Materials and Methods

### 3D MSLA Printer, Print Settings and CAD Designs

The 3D printer used for these experiments was the Prusa SL1S Speed MSLA printer. This Printer has a high-resolution Monochrome LCD screen that displays the layers onto the print bed and a UV LED panel that cures the resin at a 405 nm wavelength.

All CAD designs were made using SolidWorks, and the slicing software used was the Prusa Slicer. All slicing was adjusted at a layer height of 0.01 μm, and a layer curing time of 60 seconds.

### Rheology of 3D Printed Hydrogel

To determine the mechanical properties of the 3D printed hydrogels, a SmartPave 92 Dynamic Shear Rheometer (Anton Paar, Austria) was used. Four different prints of the MeHA and the MeHA 3TAA were tested immediately after printing. A circle design of 1 mm in thickness and 10 mm in diameter was used for the test.

Immunostaining of 3D Printed iPSC Cardiomyocytes

Immunostaining of 3D printed MeHA 3TAA with iPSC cardiomyocytes was done to evaluate the effectiveness of 3D printing tissue models and its effect in live cells. The procedure was done by printing discs of 3 cm in diameter and 10 mm in thickness, with MeHA 3TAA resin and iPSC cardiomyocytes. After printing, the gels were transferred to Transwell inserts in 12-well plates and cultured for 5 days in RPMI B27 medium.

Following culturing, the cells were fixed with PFA, and permeabilized using 0.1% triton. They were first stained using CTnT in solution with BSA to prevent non-specific binding, and later stained with F-Actin and DAPI. For a reference of the stains and their use, please refer to Table 1.

### Results and Discussion

3D printing of MeHA 3TAA can be performed using a Prusa SL1S printer. Precision and fidelity are achieved in translating the hydrogel into a 3D SLA printable material.

### Rheology of 3D Printed MeHA 3TAA

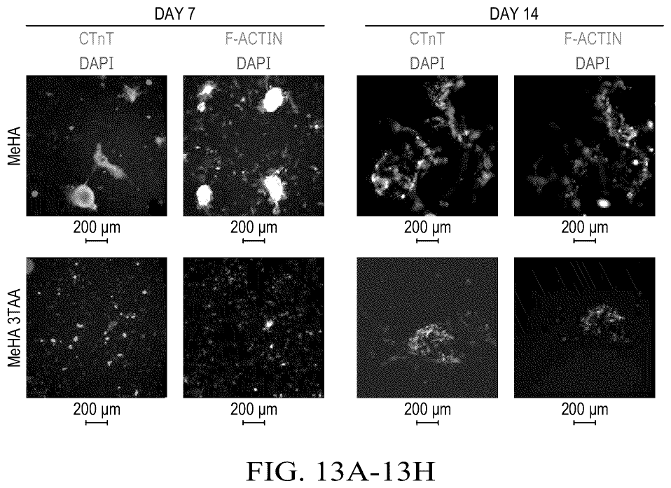

FIG. 14 shows a visual representation of the statistical comparison that showcases a transformation in the mechanical properties of the MeHA 3TAA after the 3D printing process. An examination of the storage and loss modulus, detailed in Table 5 for average values, underscores a substantial divergence between the non-3D printed and 3D printed hydrogels. This disparity signifies a marked enhancement in strength resulting from the 3D printing of MeHA 3TAA. These implications are profound, especially in the context of introducing engineered tissue into the native myocardium.

TABLE 5
Average measurements of complex modulus for
3D-printed and non-3D printed MeHA 3TAA.

| | | Measurement | Standard | Coefficient of |
|---|---|---|---|---|
| Hydrogel | Modulus | (Pa) | Deviation (SD) | Variation (%) |
| MeHA 3TAA | Storage | 26.7 | 6.9 | 25.9 |
| 3D printed MeHA 3TAA | Storage | 46.6 | 1.85 | 3.98 |
| MeHA 3TAA | Loss | 14.7 | 3.40 | 23.1 |
| 3D printed MeHA 3TAA | Loss | 8.74 | 1.09 | 12.5 |

The significance of this discovery becomes even more apparent when considering the potential implications for modulating the mechanical characteristics of the hydrogel. The ability to strengthen the material through 3D printing stands as an important breakthrough, opening avenues for refined control over the material's strength, which remains an important factor when integrating engineered tissue into the intricate environment of the native myocardium.

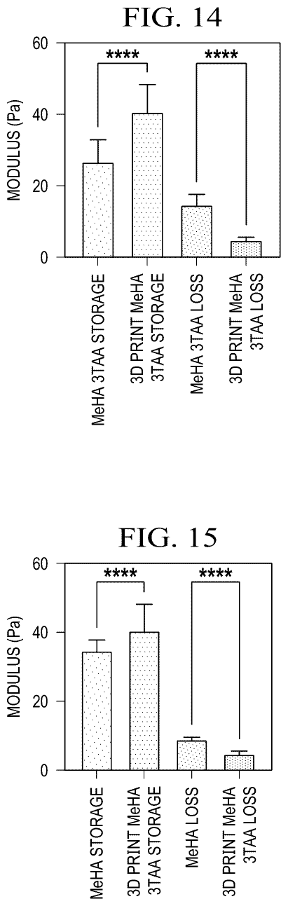

Taking this exploration further, FIG. 15 shows a statistical comparison between the original MeHA and the 3D printed MeHA 3TAA. Notably, the introduction of the 3TAA induces a discernible loss in strength in the biomaterial, as was seen in the previous study. This phenomenon is not only reversed but actually improved upon after the 3D printing process. The depicted significant differences in storage and loss modulus between the two materials accentuates the transformative impact of 3D printing, with the 3D printed material showing g higher values in storage modulus and lower values in loss modulus. This evidence underscores the multifaceted benefits of 3D printing, not only amplifying the material strength but also fin-tuning its mechanical properties in a manner that holds immense promise for advancing tissue engineering.

3D Printing of MeHA 3TAA with iPSC Cardiomyocytes

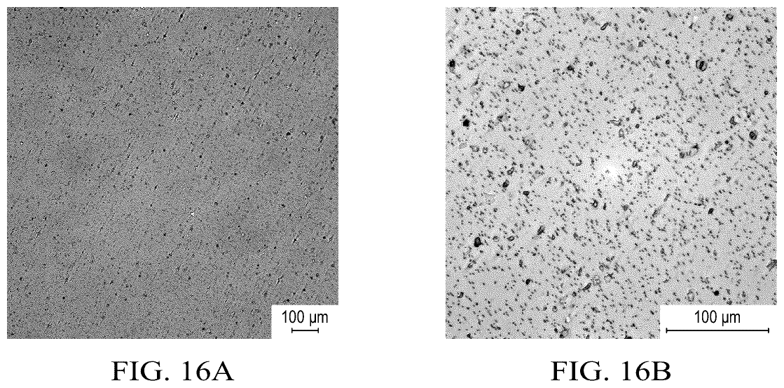

The 3D printing of the MeHA 3TAA with iPSC-derived cardiomyocytes can be done in sterile conditions inside a cell culture hood. Post-print analysis, as illustrated in FIGS. 16A-16B, reveals a discernible cellular directionality, suggesting an alignment of the cells that was facilitated by the 3D printing process. This alignment appears to occur in a single-line fashion, corresponding to the printer's layer by layer approach. The single-lines are substantially aligned, approximately parallel to one another, and approximately normal to planes defined by the layers.

This observed cell orientation bears significance in the realm of cardiac tissue engineering, presenting a distinct advantage over other manufacturing processes. The intricate structure and organization of myocytes significantly influence both the electrical propagation and mechanical properties of the heart. The apparent ability of 3D printing to arrange cells in a specific direction underscores its potential to precisely mimic the native architecture of cardiac tissue. This inherent capability positions 3D printing as a compelling technology for advancing cardiac tissue engineering.

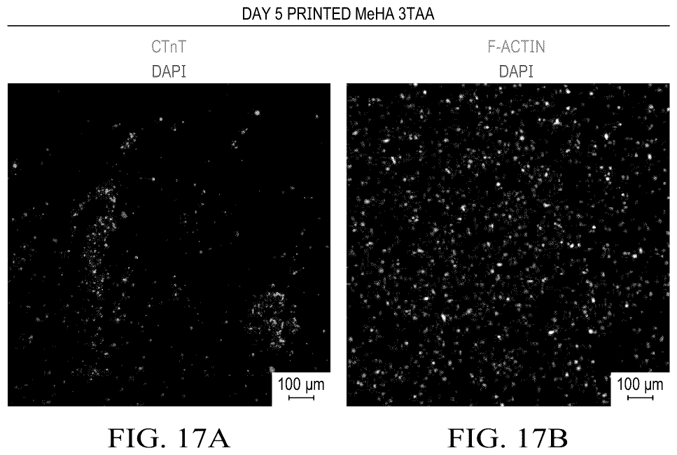

Furthermore, FIGS. 17A-17B provide additional insights, showcasing the immunostaining for day 5 of the 3D printed iPSC cardiomyocytes encapsulated in MeHA 3TAA hydrogels. The figure illustrates that 3D printing the hydrogel with cells does not compromise their culturing and growth. This evidence reinforces the robust compatibility of the 3D printing process with cellular viability, affirming its potential for seamlessly integrating living cells into complex tissue structures. These findings further bolster the argument for the efficacy of 3D printing in cardiac tissue engineering, emphasizing not only its architectural precision but also its biocompatibility, crucial for the successful development of functional and physiologically relevant cardiac constructs.

### Conclusions

The transformative impact of 3D printing technologies on tissue engineering is undeniable, ushering in crucial advancements that have propelled the field to new heights. One of the most remarkable feats enabled by this technology is the ability to 3D print full-size models of organs, such as the heart, marking a paradigm shift in our approach to replicating complex anatomical structures. The unparalleled advantages of 3D printing are due to its capacity for high-throughput, high-resolution, and reproducible tissue constructs that can be finely tuned to meet patient-specific needs. While these technological strides have surpassed expectations, a critical frontier in the domain of cardiac tissue engineering lies in the exploration of suitable materials for use with 3D printing technologies.

Constructing cardiac tissues that authentically replicate the intricate environment of the heart presents an ongoing challenge. The ideal materials for this purpose must not only be mechanically tunable but also possess electrical conductivity. Studies indicate that materials exhibiting electrical conductivity outperform their counterparts, demonstrating enhanced adhesion, alignment, organization, and maturation of cardiomyocytes.

In this study, a novel Methacrylate Hyaluronic Acid hydrogel, conjugated with 3-thiopheneacetic acid, emerges as a promising candidate for 3D stercolithography (SLA) printing. Leveraging a 3D SLA Prusa MSLA printer operating at a wavelength of 504 nm, the hydrogel consistently yielded solid and stable prints. Rheological data revealed that the young's modulus of these 3D printed hydrogels surpassed that of constructs produced without a 3D printer, suggesting the potential for tunable mechanical properties through 3D printing.

Moreover, the hydrogel was strategically combined with induced pluripotent stem cell (iPSC)-derived cardiomyocytes to assess its compatibility with biological samples. The resulting hydrogels exhibited a distinctive cellular arrangement, hinting at the printer's automatic alignment of cells in a directional pattern, mirroring the cellular organization found in native tissues. Immunostaining imaging further affirmed the resilience of the cells through the printing process, highlighting their retained biological activity.

In conclusion, this study underscores the feasibility of 3D SLA printing for a conductive hydrogel, showcasing superior properties compared to hydrogels developed without the aid of 3D printing. The findings not only contribute to the evolving landscape of cardiac tissue engineering but also spotlight the potential of 3D printing technologies to revolutionize the fabrication of functional and biocompatible tissue constructs.

The term compound is intended to mean a substance formed when two or more chemical elements are chemically bonded together, the elements present in ratios with a limited range of variation and characteristic crystal structure. The term phase is intended to mean a limited range of compositions of a mixture of the elements (in a thermochemical system) throughout which the chemical potential of the mixture varies with composition, and which either changes discontinuously or remains constant outside of that range.

The term uniformly is intended to mean unvarying or deviating very little from a given and/or expected value (e.g., within 10% of). The term substantially is intended to mean largely but not necessarily wholly that which is specified. The term approximately is intended to mean at least close to a given value (e.g., within 10% of). The term generally is intended to mean at least approaching a given state. The term coupled is intended to mean connected, although not necessarily directly, and not necessarily mechanically. The term proximate, as used herein, is intended to mean close, near adjacent and/or coincident; and includes spatial situations where specified functions and/or results (if any) can be carried out and/or achieved. The term distal, as used herein, is intended to mean far, away, spaced apart from and/or non-coincident, and includes spatial situation where specified functions and/or results (if any) can be carried out and/or achieved. The term deploying is intended to mean designing, building, shipping, installing and/or operating.

The terms first or one, and the phrases at least a first or at least one, are intended to mean the singular or the plural unless it is clear from the intrinsic text of this document that it is meant otherwise. The terms second or another, and the phrases at least a second or at least another, are intended to mean the singular or the plural unless it is clear from the intrinsic text of this document that it is meant otherwise. Unless expressly stated to the contrary in the intrinsic text of this document, the term or is intended to mean an inclusive or and not an exclusive or. Specifically, a condition A or B is satisfied by any one of the following: A is true (or present) and B is false (or not present), A is false (or not present), and B is true (or present), and both A and B are true (or present). The terms a and/or an are employed for grammatical style and merely for convenience.

The term plurality is intended to mean two or more than two. The term any is intended to mean all applicable members of a set or at least a subset of all applicable members of the set. The phrase any integer derivable therein is intended to mean an integer between the corresponding numbers recited in the specification. The phrase any range derivable therein is intended to mean any range within such corresponding numbers. The term means, when followed by the term “for” is intended to mean hardware, firmware and/or software for achieving a result. The term step, when followed by the term “for” is intended to mean a (sub) method, (sub) process and/or (sub) routine for achieving the recited result. Unless otherwise defined, all technical and scientific terms used herein have the same meaning as commonly understood by one of ordinary skill in the art to which this present disclosure belongs. In case of conflict, the present specification, including definitions, will control.

The described embodiments and examples are illustrative only and not intended to be limiting. Although embodiments of the present disclosure can be implemented separately, embodiments of the present disclosure may be integrated into the system(s) with which they are associated. All the embodiments of the present disclosure disclosed herein can be made and used without undue experimentation in light of the disclosure. Embodiments of the present disclosure are not limited by theoretical statements (if any) recited herein. The individual steps of embodiments of the present disclosure need not be performed in the disclosed manner, or combined in the disclosed sequences, but may be performed in any and all manner and/or combined in any and all sequences. The individual components of the embodiments of the present disclosure need not be formed in the disclosed shapes, or combined in the disclosed configurations, but could be provided in any and all shapes, and/or combined in any and all configurations. The individual components need not be fabricated from the disclosed materials but could be fabricated from any and all suitable materials. Homologous replacements may be substituted for the substances described herein. Agents which are both chemically and physiologically related may be substituted for the agents described herein where the same or similar results would be achieved.

Various substitutions, modifications, additions and/or rearrangements of the features of embodiments of the present disclosure may be made without deviating from the scope of the underlying inventive concept. All the disclosed elements and features of each disclosed embodiment can be combined with, or substituted for, the disclosed elements and features of every other disclosed embodiment except where such elements or features are mutually exclusive. The scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” or “mechanism for” or “step for”. Sub-generic embodiments of this disclosure are delineated by the appended independent claims and their equivalents. Specific embodiments of this disclosure are differentiated by the appended dependent claims and their equivalents.

What is claimed is:

1. A composition of matter, comprising:
a conductive biopolymer comprising a plurality of layers, each of the plurality of layers comprising
methacrylated hyaluronic acid conjugated with 3-thiophene acetic acid; and
myocytes.

2. The composition of matter of claim 1, wherein the myocytes comprise iPSC-derived cardiomyocytes.

3. The composition of matter of claim 1, wherein the myocytes are substantially aligned in single lines.

4. The composition of matter of claim 3, wherein the single lines are approximately parallel to one another and approximately normal to planes defined by the plurality of layers.

5. The composition of matter of claim 1, further comprising Lithium Phenyl-2,4,6-trimethylbenzoylphosphinate.

6. The composition of matter of claim 1, further comprising iodine.

7. The composition of matter of claim 1, further comprising 1,1′-Carbonyldiimidazole.

8. A composition of matter, comprising:
a conductive biopolymer comprising a plurality of layers, each of the plurality of layers comprising
methacrylated hyaluronic acid conjugated with poly(3-thiophene) acetic acid; and
myocytes.

9. The composition of matter of claim 8, wherein the myocytes comprise iPSC-derived cardiomyocytes.

10. The composition of matter of claim 8, wherein the myocytes are substantially aligned in single lines.

11. The composition of matter of claim 10, wherein the single lines are approximately parallel to one another and approximately normal to planes defined by the plurality of layers.

12. The composition of matter of claim 8, further comprising Lithium Phenyl-2,4,6-trimethylbenzoylphosphinate.

13. The composition of matter of claim 8, further comprising iodine.

14. The composition of matter of claim 8, further comprising 1,1′-Carbonyldiimidazole.

15. A method, comprising 3D printing a conductive biopolymer comprising a plurality of layers, each of the plurality of layers comprising
methacrylated hyaluronic acid conjugated with at least one of 3-thiophene acetic acid and poly(3-thiophene) acetic acid; and
myocytes.

16. The method of claim 15, wherein the myocytes comprise iPSC-derived cardiomyocytes.

17. The method of claim 15, wherein the myocytes are substantially aligned in single lines.

18. The method of claim 17, wherein the single lines are approximately parallel to one another and approximately normal to planes defined by the plurality of layers.

19. The method of claim 15, wherein each of the plurality of layers comprises Lithium Phenyl-2,4,6-trimethylbenzoylphosphinate.

20. The method of claim 15, wherein each of the plurality of layers comprises 1,1′-Carbonyldiimidazole.
